(19) United States
(12) Patent Application Publication
(10) Pub. No.: US 2013/0296522 A1
(43) Pub. Date: Nov. 7, 2013

(54) POLYURETHANE USEFUL FOR ORTHOPEDICS EXTERNAL FIXING SYSTEM IN COMPLEX ENVIRONMENT AND PREPARATION METHOD THEREOF

(71) Applicant: Xieping Zhu, Shanghai (CN)

(72) Inventor: Xieping Zhu, Shanghai (CN)

(21) Appl. No.: 13/814,503

(22) PCT Filed: Jun. 2, 2010
(86) PCT No.: PCT/CN2010/000785

§ 371 Date: Feb. 5, 2013

(30) Foreign Application Priority Data
Jun. 2, 2009 (CN) 200910052379.X

(51) Int. Cl. A61L 15/12

(52) U.S. Cl. CPC A61L 15/12
USPC 528/60

(57) ABSTRACT

A polyurethane used for orthopedics external fixing system in complex environment is obtained by reaction of mixture of polyether polyol with modified isocyanate in the weight ratio of 1:0.6-0.75. The modified isocyanate is obtained by reaction of polyphenyl polymethylene isocyanate with polyether diol. The components and corresponding weight percents of the mixture of polyether polyol are as follows: 60-65% polyether polyol as shown by formula (I), in the formula, m is an integer of 7-15, R represents mannitol, sucrose or pentaerythritol; 0.3-1% catalyst; 0.1-2% foam stabilizer; 0.1-2% or 3-20% foaming agent; 25-40% filler and 0.5-2% functional auxiliary agent. The method of producing the polyurethane is also provided.

### CROSS-REFERENCE TO RELATED APPLICATIONS

This application is a National Stage Appl. filed under 35 USC 371 of International Patent Application No. PCT/CN2010/000785 with an international filing date of Jun. 2, 2010, designating the United States, and further claims priority benefits to Chinese Patent Application No. 200910052379.X filed Jun. 2, 2009. Inquiries from the public to applicants or assignees concerning this document or the related applications should be directed to: Matthias Scholl P. C., Attn.: Dr. Matthias Scholl Esq., 14781 Memorial Drive, Suite 1319, Houston, Tex. 77079.

### FIELD OF THE INVENTION

The invention relates to a polymer material and a preparation method thereof, and more particularly to a polyurethane for an external fixation system for bone surgeries and a method for preparing the same.

### BACKGROUND OF THE INVENTION

Chinese Patent Application No. 200810038254.7, titled as a new external fixation system for bone surgeries, a using method thereof, and a polyurethane for the fixation system, has disclosed a polyurethane for an external fixation system for bone surgeries. However, in poor conditions, for example, in the field at a low temperature, where no hospitals are available, and the temperature is about 5-15° C., the fixation system cannot be hardened by the polyurethane; whereas in the temperature of 25-30° C., the hardening of the fixation system is too fast and produces too much heat.

### SUMMARY OF THE INVENTION

In view of the above-described problems, it is one objective of the invention to provide a polyurethane for an external fixation system for bone surgeries. The polyurethane can effectively overcome problems resulting from hardening and heat production.

It is another objective of the invention to provide a method for preparing the polyurethane for an external fixation system for bone surgeries.

Technical scheme of the invention is as follows:

A polyurethane for an external fixation system for bone surgeries, is a polyether type polyurethane represented by a formula of

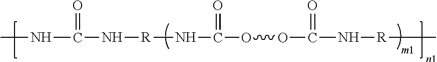

in which, ml represents an integer from 5 to 20, nl represents an integer from 7 to 15 or from 60 to 120, R represents a phenyl group, and

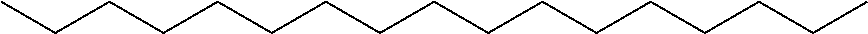 [SMILES: CCCCCCCCCCCCCCCCC]

represents a polyether polyol.

The polyether type polyurethane is prepared by using a polyether polyol mixture and a modified isocyanate as raw materials according to a weight ratio of 1:0.6-0.75.

The polyether polyol mixture comprises ingredients as follows:

| | |
|---|---|
| the polyether polyol | 60-65 wt. %, |
| a catalyst | 0.3-1 wt. %, |
| a foam stabilizer | 0.1-2 wt. %, |
| a foaming agent | 0.1-2 wt. % or 3-20 wt. %; |
| a filler | 25-40 wt. %; and |
| a functional additive | 0.5-2 wt. %. |

The polyether polyol is represented by a formula of

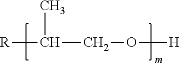

in which, m is an integer from 7 to 15, and R represents mannite, sucrose, or pentaerythritol.

The modified isocyanate has a formula of

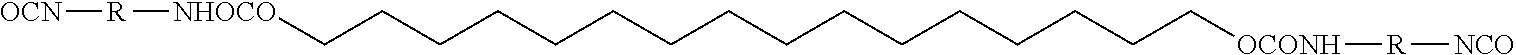

in which, R represents methyl, benzene ring, a carbamate, or a polyester,

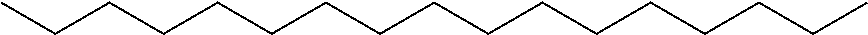 [SMILES: CCCCCCCCCCCCCCCCC]

represents a polyether glycol.

The modified isocyanate is prepared by using a polyphenylpolymethylene isocyanate (liquefied 4,4′-diphenylmethane diisocyanate (MDI)) and the polyether glycol as raw materials. The polyphenylpolymethylene isocyanate is represented by a formula of

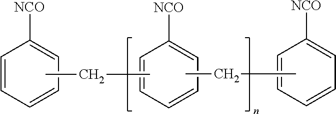

in which, n represents 1, 2, or 3.

The catalyst in the polyether polyol mixture is selected from dimethyl ethylenimine, dimethyl ethanolamine, or triethylene diamine.

The foam stabilizer in the polyether polyol mixture is a silicone stabilizer.

The foaming agent in the polyether polyol mixture is water or CH₃CCl₂F.

If the foaming agent is water, the content of the foaming agent is 0.1-2 wt. % of the polyther polyol mixture; and if the foaming agent is CH₃CCl₂F, the content of the foaming agent is 3-20 wt. % of the polyther polyol mixture.

The filler in the polyther polyol mixture is calcium carbonate, or barium sulfate.

The functional additive in the polyther polyol mixture is silica.

A method for prepare the polyurethane, comprises steps as follows:
a) mixing the polyphenylpolymethylene isocyanate and the polyether glycol, and allowing for reaction at a temperature of 70-90° C. to obtain the modified isocyanate;
b) mixing the polyether polyol, the catalyst, the foam stabilizer, the foaming agent, the filler, and the functional additive according to a certain ratio to obtain the polyether polyol mixture; and
c) mixing the polyether polyol mixture from step b) and the modified isocyanate from step a) according to a ratio of 1:0.6-0.75 to obtain the polyurethane.

Raw materials used in the preparation are all commercial products.

The polyurethane of the invention can be used at a temperature of 5-15° C.

The polyurethane of the invention can be used at a temperature of 25-30° C.

An optimal temperature for using the polyurethane of the invention is 21-25° C.

Advantages of the invention are summarized as follows:
The polyurethane of the invention can be used at a temperature of 5-15° C., and can fix fracture regions that are difficult to fix by plasters. The polyurethane has sufficient rigidity and intensity. X-ray can penetrate the polyurethane, so that the situation of the fracture region can be known. Besides, the polyurethane is sanitary and is convenient to expose the skin to oxygen; medical personnel can easily take off the polyurethane to clean and process the skin on the fracture region. Furthermore, the polyurethane has a much lower cost than metal stents, a much better fixation effect than polymer bandages or plasters, and does not result in infections or secondary hurts. Finally, when the polyurethane is attached to the skin, a temperature of a heat production is no higher than 38° C., and different shaping durations can be selected to avoid inducing cardiovascular diseases.

### DETAILED DESCRIPTION OF THE EMBODIMENTS

To further illustrate the invention, experiments detailing a polyurethane and a method for preparing the same are described below. It should be noted that the following examples are intended to describe and not to limit the invention.

### EXAMPLE 1

Preparation of a modified isocyanate: a polyphenylpolymethylene isocyanate and a polyether glycol were mixed, and a reaction was carried out at a temperature of 70-90° C. to obtain the modified isocyanate.

Preparation of a polyether polyol mixture: 60 weight parts of a polyether polyol, 0.5 weight part of triethylene diamine, 1 weight part of a silicone stabilizer, 0.5 weight part of water, 37 weight parts of calcium carbonate, and 1 weight part of silica were collected and mixed to obtain the polyether polyol mixture.

Preparation of a polyurethane: the polyether polyol mixture and the modified isocyanate are mixed according to a ratio of 1:0.6 to obtain the polyurethane.

### EXAMPLE 2

Preparation of a modified isocyanate is the same as that of Example 1.

Preparation of a polyether polyol mixture: 61 weight parts of a polyether polyol, 1 weight part of triethylene diamine, 2 weight parts of a silicone stabilizer, 0.1 weight part of water, 35.4 weight parts of calcium carbonate, and 0.5 weight part of silica were collected and mixed to obtain the polyether polyol mixture.

Preparation of a polyurethane: the polyether polyol mixture and the modified isocyanate are mixed according to a ratio of 1:0.65 to obtain the polyurethane.

### EXAMPLE 3

Preparation of a modified isocyanate is the same as that of Example 1.

Preparation of a polyether polyol mixture: 62 weight parts of a polyether polyol, 0.3 weight part of triethylene diamine, 2 weight parts of a silicone stabilizer, 2 weight parts of water, 33 weight parts of calcium carbonate, and 0.7 weight part of silica were collected and mixed to obtain the polyether polyol mixture.

Preparation of a polyurethane: the polyether polyol mixture and the modified isocyanate are mixed according to a ratio of 1:0.65 to obtain the polyurethane.

### EXAMPLE 4

Preparation of a modified isocyanate is the same as that of Example 1.

Preparation of a polyether polyol mixture: 63 weight parts of a polyether polyol, 1 weight part of dimethyl ethanolamine, 0.5 weight part of a silicone stabilizer, 1 weight part of water, 33 weight parts of calcium carbonate, and 1.5 weight parts of silica were collected and mixed to obtain the polyether polyol mixture.

Preparation of a polyurethane: the polyether polyol mixture and the modified isocyanate are mixed according to a ratio of 1:0.7 to obtain the polyurethane.

### EXAMPLE 5

Preparation of a modified isocyanate is the same as that of Example 1.

Preparation of a polyether polyol mixture: 64 weight parts of a polyether polyol, 0.5 weight part of dimethyl ethylenimine, 1.5 weight parts of a silicone stabilizer, 7 weight parts of CH₃CCl₂F, 25 weight parts of barium sulfate, and 2 weight parts of silica were collected and mixed to obtain the polyether polyol mixture.

Preparation of a polyurethane: the polyether polyol mixture and the modified isocyanate are mixed according to a ratio of 1:0.75 to obtain the polyurethane.

### EXAMPLE 6

Preparation of a modified isocyanate is the same as that of Example 1.

Preparation of a polyether polyol mixture: 65 weight parts of a polyether polyol, 1 weight part of dimethyl ethylenimine, 1.2 weight parts of a silicone stabilizer, 1 weight part of water, 30 weight parts of barium sulfate, and 1.8 weight parts of silica were collected and mixed to obtain the polyether polyol mixture.

Preparation of a polyurethane: the polyether polyol mixture and the modified isocyanate are mixed according to a ratio of 1:0.63 to obtain the polyurethane.

### EXAMPLE 7

Preparation of a modified isocyanate is the same as that of Example 1.

Preparation of a polyether polyol mixture: 60 weight parts of a polyether polyol, 0.3 weight part of dimethyl ethylenimine, 0.2 weight part of a silicone stabilizer, 14 weight parts of CH₃CCl₂F, 25 weight parts of barium sulfate, and 0.5 weight part of silica were collected and mixed to obtain the polyether polyol mixture.

Preparation of a polyurethane: the polyether polyol mixture and the modified isocyanate are mixed according to a ratio of 1:0.68 to obtain the polyurethane.

The invention claimed is:

1. A polyurethane for an external fixation system for bone surgeries, being a polyether type polyurethane represented by a formula of 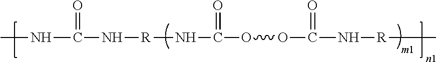
in which, ml representing an integer from 5 to 20, nl representing an integer from 7 to 15 or from 60 to 120, R representing a phenyl group, and 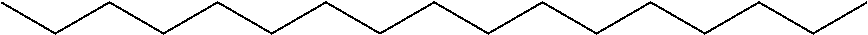 [SMILES: CCCCCCCCCCCCCCCCC]
representing a polyether polyol;
wherein
the polyether type polyurethane is prepared by using a polyether polyol mixture and a modified isocyanate as raw materials according to a weight ratio of 1:0.6-0.75;
(1) the polyether polyol mixture comprises ingredients as follows:

| | |
|---|---|
| the polyether polyol | 60-65 wt. %, |
| a catalyst | 0.3-1 wt. %, |
| a foam stabilizer | 0.1-2 wt. %, |
| a foaming agent | 0.1-2 wt. % or 3-20 wt. %; |
| a filler | 25-40 wt. %; and |
| a functional additive | 0.5-2 wt. %; |

the polyether polyol being represented by a formula of 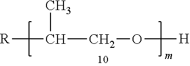
in which, m is an integer from 7 to 15, R represents mannite, sucrose, or pentaerythritol; and
(2) the modified isocyanate has a formula of 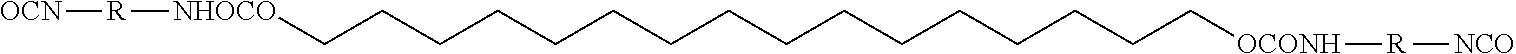
in which, R represents methyl, benzene ring, a carbamate, or a polyester, 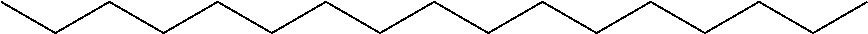 [SMILES: CCCCCCCCCCCCCCCCC]
represents a polyether glycol.

2. The polyurethane of claim 1, wherein the modified isocyanate is prepared by using a polyphenylpolymethylene isocyanate and the polyether glycol as raw materials.

3. The polyurethane of claim 1, wherein the catalyst in the polyether polyol mixture is selected from dimethyl ethylenimine, dimethyl ethanolamine, or triethylene diamine.

4. The polyurethane of claim 1, wherein the foam stabilizer in the polyether polyol mixture is a silicone stabilizer.

5. The polyurethane of claim 1, wherein the foaming agent in the polyether polyol mixture is water or CH₃CCl₂F.

6. The polyurethane of claim 5, wherein
if the foaming agent is water, the content of the foaming agent is 0.1-2 wt. % of the polyther polyol mixture; and
if the foaming agent is CH₃CCl₂F, the content of the foaming agent is 3-20 wt. % of the polyther polyol mixture.

7. The polyurethane of claim 1, wherein the filler in the polyther polyol mixture is calcium carbonate, or barium sulfate.

8. The polyurethane of claim 1, wherein the functional additive in the polyther polyol mixture is silica.

9. A method for preparing the polyurethane of claim 1, comprising the steps of:
a) mixing the polyphenylpolymethylene isocyanate and the polyether glycol, and allowing for reaction at a temperature of 70-90° C. to obtain the modified isocyanate;
b) mixing the polyether polyol, the catalyst, the foam stabilizer, the foaming agent, the filler, and the functional additive according to a certain ratio to obtain the polyether polyol mixture; and
c) mixing the polyether polyol mixture from step b) and the modified isocyanate from step a) according to a ratio of 1:0.6-0.75 to obtain the polyurethane.
